(19) United States
(12) Patent Application Publication
(10) Pub. No.: US 2026/0234546 A1
(43) Pub. Date: Aug. 13, 2026

(54) Storage Stable Lacticaseibacillus Rhamnosus

(71) Applicant: CHR. HANSEN A/S, Hoersholm (DK)

(72) Inventors: Han HUI, Beijing (CN); Stina HOFF, Vaerloese (DK); Vera Kuzina POULSEN, Vaerloese (DK); Solvej PRECHT, Fredensborg (DK)

(73) Assignee: CHR. HANSEN A/S, Hoersholm (DK)

(21) Appl. No.: 19/475,242

(22) PCT Filed: Apr. 18, 2024
(86) PCT No.: PCT/EP2024/060570

§ 371 Date: Oct. 14, 2025

(30) Foreign Application Priority Data
Apr. 20, 2023 (EP) 23168873.0

(51) Int. Cl. C12N 1/20; A23C 9/123

(52) U.S. Cl. CPC C12N 1/20; A23C 9/1234

(57) ABSTRACT

The present disclosure or application provides bacteria of the species Lacticaseibacillus rhamnosus (j_. rhamnosus) DSM 34194. The bacteria are able to maintain a high viability in fermented milk products during storage even at ambient temperature and are further characterized by a low post-acidification activity. The disclosure or application further provides composition or starter cultures comprising the bacteria, methods of producing fermented milk products using the bacteria or starter culture and fermented milk products obtainable by these methods.

### TECHNICAL FIELD

The present disclosure or application relates to bacteria of the species Lacticaseibacillus rhamnosus (L. rhamnosus) which maintain a high viability in fermented milk products during storage even at ambient temperature. The bacteria of the present disclosure or application are further characterized by a low post-acidification activity. Loss of viability and post-acidification are effects frequently observed in fermented milk products especially in case of storage at ambient temperatures, i.e., if the cold chain is broken. The disclosure or application provides bacteria of the species L. rhamnosus, starter cultures comprising the bacteria, methods of producing fermented milk products using the bacteria or starter culture and fermented milk products obtainable by these methods.

### BACKGROUND

Fermented milk products are nutritional and delicious dairy foods which are produced and consumed worldwide. Bacterial cultures commonly used for fermenting milk and for the production of dairy foods comprise lactic acid bacteria such as Lactobacillus delbrueckii subsp. bulgaricus and Streptococcus thermophilus. The bacteria produce lactic acid and other compounds, in particular numerous further organic acids, which cause a reduction of the pH and provide a delicate acidic flavor as well as a creamy thick texture. Consumers appreciate the balanced flavors and the benefits associated with consuming yogurt with live and active bacterial cultures.

The number of viable lactic acid bacteria in a fermented milk product is routinely determined for a given product. In many countries, there is also a minimum total cell count requirement for viable lactic acid bacteria in fermented milk products. For example, in China fermented milk should maintain a total lactic acid bacteria cell count of at least 10⁶ CFU/g throughout shelf life.

This can be a problem, as Lactobacillus delbrueckii and Streptococcus thermophilus which are most frequently used for producing fermented milk products are unstable when stored at ambient temperatures and to a lesser extent even when stored at temperatures around 4° C. They may additionally have different performances in different milk recipes.

Maintaining viability of lactic acid bacteria in fermented milk products is commonly achieved by cooling the product. For example, in the production of many dairy products, such as yogurt, fermentation of the product is stopped at a desired pH or desired total acid level (TA) by cooling. While continuous cooling preserves viability, it does not completely prevent metabolic activity of the bacteria during storage.

In many countries, conditions for distributing and selling fermented milk products are not ideal. This is due to the fact that the refrigeration temperature is not maintained throughout the shelf life of the product. As a consequence of the loss of cold chain protection viable lactic acid bacteria exhibit increased metabolic activity which causes further acidification of the fermented milk product, also known as post-acidification, and a loss of viability of the lactic acid bacteria. Fermented milk products stored for 14 days or more at a temperature of 25° C. or more usually contain a total lactic acid bacteria cell count of less than 10⁶ CFU/g, often significantly less than 10⁶ CFU/g. Respective products no longer meet the threshold for viable cell count required by many states. Further, the quality of the fermented milk product declines in which case the consumer will likely have a negative experience. These problems of loss of viability and flavor are particularly pertinent for products fermented with Lactobacillus delbrueckii subsp. bulgaricus and Streptococcus thermophilus.

To solve these problems, lactic acid bacteria with strong viability, low or no additional post-acidification activity and low sensory impact on the fermented milk product are needed. In particular, respective bacteria would have to maintain viability when stored under ambient conditions and at the same time could not produce undesired flavors, further pH reduction or gas production.

Bacterial species such as Lactobacillus paracasei, Lactobacillus plantarum and Lactobacillus rhamnosus are known to survive well at ambient temperatures. However, strains of these species tend to contribute significantly to post-acidification when metabolic activity increases in the presence of ambient temperatures.

For example, WO2019/081577 of Chr. Hansen A/S discloses L. rhamnosus strain DSM 32666 and mutants thereof. WO2019/081577 teaches the use of CBS141584 as part of a starter culture to provide an enhanced creamy flavor to a fermented product. However, post-acidification effects are not mentioned. Similarly, WO2021/239574 of Chr. Hansen A/S discloses L. rhamnosus strain DSM 33515 and mutants thereof which exhibit low post-acidification activity.

It would also be desirable to provide starter cultures that address the viability and post-acidification challenges in fermented milk products, preferably those that are prepared from milk base containing sugars such as sucrose or fructose.

### SUMMARY

The above problem is now solved by bacteria of the species L. rhamnosus deposited as DSM 34194 and mutants thereof.

The present disclosure or application provides a solution to the above problem by providing a new and improved robust L. rhamnosus strain that maintains high viability even under ambient temperatures and provides very low post-acidification activity.

The present disclosure or application also provides a bacterium of the species L. rhamnosus, wherein the bacterium is a mutant of the bacterium deposited as DSM 34194, wherein the mutant maintains viability during storage at 25° C., and wherein the test for maintaining viability comprises storing a fermented milk product comprising at least 1×10⁷ CFU/g of the mutant before storage and wherein the fermented milk product comprises at least 1×10⁶ CFU/g of the mutant after storage over 21 days at 25° C.

The disclosure or application also provides a composition comprising bacteria of the species L. rhamnosus as described above. In one embodiment, the composition comprises bacteria of the species L. rhamnosus as described above in a concentration of at least 1×10⁹ CFU/g. In a preferred embodiment, the composition comprises bacteria of the species L. rhamnosus in a concentration of at least 1×10¹⁰ CFU/g. In an even more preferred embodiment, the composition comprises bacteria of the species L. rhamnosus in a concentration of at least 1×10¹¹ CFU/g.

In a further embodiment, the above composition comprises a starter culture. In another embodiment, the starter culture comprises bacteria of the species Lactobacillus delbrueckii subsp. bulgaricus and/or Streptococcus thermophilus.

The composition of the present disclosure or application may additionally comprise cryoprotectants, lyoprotectants, antioxidants, nutrients, fillers, flavorings, flavors or mixtures thereof. The composition may be in frozen or freeze-dried form.

In a related embodiment, the disclosure or application also provides a method of producing a fermented milk product comprising adding the bacterium of the species L. rhamnosus as described above or the composition as described above to a milk base and fermenting the milk base at a temperature between 22° C. and about 43° C. until a pH of 4.5 or less or a total acidity (TA) of 70 or less is reached or until a pH of 4.7 or less, such as 4.55 or less, is reached.

The present disclosure or application also provides fermented milk products comprising bacteria of the species L. rhamnosus as described above. In one embodiment, the milk product comprises bacteria of the species L. rhamnosus as described above in a concentration of at least 1×10⁶ CFU/g. The milk product can be obtainable by the method as described above.

In another embodiment, the fermented milk product can maintain a pH above 3.5 when stored for at least 21 days at 25° C. In a preferred aspect, the fermented milk product maintains a viable lactic acid bacteria cell count of at least 1×10⁶ CFU/g when stored for at least 21 days at 25° C.

The present disclosure or application also provides fermented milk products as described above, wherein the fermented milk product is yakult, cheese, yogurt, fruit yogurt, yogurt beverage, strained yogurt (Greek yogurt, Labneh, skyr), quark, fromage frais sour cream, butter milk, white brined cheese, UF feta and cottage cheese, or cream cheese.

The present disclosure or application also provides fermented milk products as described above, wherein the fermented milk product is prepared from milk base comprising sucrose or fructose such as syrup.

### DETAILED DESCRIPTION

The present inventors surprisingly found that bacteria of the L. rhamnosus strain deposited as DSM 34194 and/or mutants thereof can advantageously be used in combination with common starter cultures for producing fermented milk products, as they combine high viability even when stored under ambient conditions for extended periods of time with low post-acidification activity. In one aspect, bacteria of the species L. rhamnosus deposited as DSM 34194 and/or mutants thereof can be characterized by maintaining viability during storage at 25° C., wherein the test for maintaining viability comprises storing a fermented milk product comprising at least 1×10⁷ CFU/g of the deposited bacteria and/or the mutant before storage and wherein the fermented milk product comprises at least 1×10⁶ CFU/g of the deposited bacteria and/or the mutant after storage over 21 days at 25° C. In a preferred aspect, the bacteria of the species L. rhamnosus deposited as DSM 34194 and/or the mutants thereof can be characterized as maintaining viability during storage at 25° C., wherein the test for maintaining viability comprises storing a fermented milk product comprising at least 1×10⁸ CFU/g, more preferably at least 1×10⁹ CFU/g, of the deposited bacteria and/or the mutant thereof before storage and wherein the fermented milk product comprises at least 1×10⁶ CFU/g of the deposited bacteria or the mutant after storage over 21 days at 25° C. In a particularly preferred aspect, the storage stability test used for determining viability after storage according to all embodiments of the present disclosure or application is carried out using stirred yogurt as the fermented milk product.

Extensive screening of 16000 mutants of a Lactobacillus rhamnosus strain led to the identification of DSM 34194. DSM 34194 was selected by high-throughput screening, based on its low post-acidification from a mutant pool generated using ethyl methanesulfonate (EMS) mutagenesis. The high-throughput screening included mutant hit picking from agar, growth of mutants in broth, milk acidification at 40° C. and post-acidification of one replicate at room temperature for 12 days and another replicate at 17° C. for 2 weeks, and selection of mutants with low post-acidification at 17° C.

In the context of the present disclosure or application, the term “lactic acid bacteria” or “LAB” is used to refer to food-grade bacteria producing lactic acid as the major metabolic end-product of carbohydrate fermentation. These bacteria are related by their common metabolic and physiological characteristics and are usually Gram-positive, low-GC, acid tolerant, non-sporulating, non-respiring, rod-shaped bacilli or cocci. During the fermentation stage, the consumption of lactose by these bacteria causes the formation 25 of lactic acid, reducing the pH and leading to the formation of a protein coagulum. These bacteria are thus responsible for the acidification of milk and for the texture of dairy products. As used herein, the term “lactic acid bacteria” encompasses, but is not limited to, bacteria belonging to the genus of Lactobacillus spp., Bifidobacterium spp., Streptococcus spp., Lacticaseibacillus spp., Lactococcus spp., such as Lactobacillus delbrueckii subsp. bulgaricus, Streptococcus thermophilus, Lactobacillus lactis, Bifidobacterium animalis, Lactococcus lactis, Lactobacillus paracasei, Lactobacillus plantarum, Lactobacillus helveticus, Lactobacillus acidophilus, Bifidobacterium breve and Leuconostoc spp.

Lacticaseibacillus rhamnosus is a lactic acid bacterium previously also known as Lactobacillus rhamnosus which has been renamed (Zheng et al., Int. J. Syst. Evol. Microbiol. DOI 10.1099/ijsem.0.004107).

In the context of the present disclosure or application, the term “mutant” refers to a strain of bacteria of the species L. rhamnosus which is obtainable from the strain deposited as DSM 34194, for example by means of genetic engineering, radiation and/or chemical treatment. It is preferred that the mutant is a functionally equivalent mutant, e.g., a mutant that has substantially the same, or improved, properties as the deposited strain in particular in relation to the effect of maintaining viability and/or inhibiting post-acidification. Respective mutants represent embodiments of the present disclosure or application. The term “mutant” in particular refers to a strain obtained by subjecting a strain of the disclosure or application to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood as one mutagenization step followed by a screening/selection step), but it is presently preferred that no more than 20, or no more than 10, or no more than 5, treatments (or screening/selection steps) are carried out. In a preferred mutant, less than 5%, or less than 1% or even less than 0.1% of the nucleotides in the bacterial genome have been replaced by another nucleotide, or deleted, in comparison to the deposited strain.

LAB are commonly added to milk in the form of a starter culture. The term “starter” or “starter culture” as used in the present context refers to a culture of one or more food-grade microorganisms, in particular to lactic acid bacteria, which are responsible for the acidification of the milk base. The “starter culture” may be Streptococcus thermophilus and/or Lactobacillus delbrueckii subsp. bulgaricus. Starter cultures may be fresh, but are most frequently frozen or freeze-dried. These products are also known as “Direct Vat Set” (DVS) cultures and are produced for direct inoculation in a fermentation vessel or vat for the production of a dairy product, such as a fermented milk product or a cheese. Respective starter cultures are commercially available from numerous sources and include Premium 1.0, F-DBA YoFlex Mild 2.0, F-DVS YF-L901, YF-907, FD-DVS CH-1, four cultures commercially available from Chr. Hansen containing mixtures of Streptococcus thermophilus and Lactobacillus delbrueckii subsp. bulgaricus.

In the context of the present disclosure or application, the term “milk” is broadly used in its common meaning to refer to liquids produced by the mammary glands of animals or by plants. In accordance with the present disclosure or application the milk may have been processed and the term “milk” includes whole milk, skim milk, fat-free milk, low fat milk, full fat milk, lactose-reduced milk, or concentrated milk. Fat-free milk is non-fat or skim, milk product. Low-fat milk is typically defined as milk that contains from about 1% to about 2% fat. Full fat milk often contains 2% fat or more. The term “milk” is intended to encompass milks from different mammal and plant sources. Mammal sources of milk include, but are not limited to, cow, sheep, goat, buffalo, camel, lama, mare and deer. Plant sources of milk include, but are not limited to, milk extracted from soybean, pea, peanut, barley, rice, oat, quinoa, almond, cashew, coconut, hazelnut, hemp, sesame seed and sunflower seed. In the methods and products of the present disclosure or application, milk derived from cows is most preferably used as a starting material for the fermentation.

The term “milk” also includes fat-reduced and/or lactose-reduced milk products. Respective products can be prepared using methods well known in the art and are commercially available. Lactose-reduced milk can be produced according to any method known in the art, including hydrolyzing the lactose by lactase enzyme to glucose and galactose, or by nanofiltration, electrodialysis, ion exchange chromatograph and centrifugation.

The term “milk product” or “milk base” is broadly used in the present disclosure or application to refer to a composition based on milk or milk components which can be used as a medium for growth and fermentation of LAB. The milk product or base comprises components derived from milk and any other component that can be used for the purpose of growing or fermenting LAB.

Prior to fermentation, the milk substrate may be homogenized and pasteurized according to methods known in the art. “Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices. “Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

The terms “fermented milk product”, “food” or “feed” product refer to products obtainable by the fermentation methods of the present disclosure or application and include yakult, cheese, yogurt, fruit yogurt, yogurt beverage, strained yogurt (Greek yogurt, Labneh, skyr), quark, fromage frais, sour cream, butter milk, white brined cheese, UF feta and cottage cheese, and cream cheese. The term food further encompasses other fermented food products, including fermented meat, such as fermented sausages, and fermented fish products.

The term “cheese” is understood to encompass any cheese, including hard, semi-hard and soft cheeses, such as cheeses of the following types: Cottage, Feta, Cheddar, Parmesan, Mozzarella, Emmentaler, Danbo, Gouda, Edam, Feta-type, blue cheeses, brine cheeses, Camembert and Brie. The person skilled in the art knows how to convert the coagulum into cheese, methods can be found in the literature, see e.g., Kosikowski, F. V., and V. V. Mistry, “Cheese and Fermented Milk Foods”, 1997, 3rd Ed. F. V. Kosikowski, L. L. C. Westport, CT. As used herein, a cheese which has a NaCl concentration below 1.7% (w/w) is referred to as a “low-salt cheese”.

In the context of the present disclosure or application, the term “yogurt” refers to products comprising Streptococcus thermophilus and Lactobacillus delbrueckii subsp. bulgaricus and optionally other microorganisms such as Lactobacillus delbrueckii subsp. lactis, Bifidobacterium animalis subsp. lactis, Lactococcus lactis, Lactobacillus acidophilus and Lactobacillus paracasei, or any microorganism derived therefrom. The lactic acid strains other than Streptococcus thermophilus and Lactobacillus delbrueckii subsp. bulgaricus, are included to give the finished product various properties, such as the property of promoting the equilibrium of the flora. As used herein, the term “yogurt” encompasses set yogurt, stirred yogurt, drinking yogurt, Petit Suisse, heat treated yogurt, strained or Greek style yogurt characterized by a high protein level and yogurt-like products.

In particular, the term “yogurt” encompasses, but is not limited to, yogurt as defined according to French and European regulations, e.g. coagulated dairy products obtained by lactic acid fermentation by means of specific thermophilic lactic acid bacteria only (i.e. Lactobacillus delbrueckii subsp. bulgaricus and Streptococcus thermophilus) which are cultured simultaneously and are found to be live in the final product in an amount of at least 10 million CFU (colony-forming unit)/g. Yogurts may optionally contain added dairy raw materials (e.g. cream) or other ingredients such as sugar or sweetening agents, one or more flavoring(s), fruit, cereals, or nutritional substances, especially vitamins, minerals and fibers, as well as stabilizers and thickeners. Optionally the yogurt meets the specifications for fermented milks and yogurts of the AFNOR NF 04-600 standard and/or the codex StanA-IIa-1975 standard. In order to satisfy the AFNOR NF 04-600 standard, the product must not have been heated after fermentation and the dairy raw materials must represent a minimum of 70% (m/m) of the finished product.

In the context of the present disclosure or application “ambient temperature” is herein defined as the range of air temperatures between 15-37° C., preferably 15-30° C., more preferably 18-25° C. or 25° C., either range or temperature at a pressure of 1 atm.

The use of the terms “a” and “an” and “the” and similar referents in the context of describing the disclosure or application (especially in the context of the claims) are to be constructed to cover both the singular and the plural, unless otherwise indicated herein or clearly contradicted by context.

In another aspect the disclosure or application provides bacteria of the species L. rhamnosus deposited as DSM 34194 or mutants thereof, wherein the bacteria maintain a higher total lactic acid cell count of a fermented milk product comprising the deposited bacterium or a mutant thereof during storage after fermentation in comparison to a milk product comprising L. rhamnosus deposited as DSM 33515, wherein the higher total lactic acid bacteria cell count is determined after storing a product fermented with a starter culture and the L. rhamnosus strain over 21 days at 25° C.

In yet another aspect the disclosure or application provides bacteria of the species L. rhamnosus deposited as DSM 34194 or mutants thereof, wherein the bacteria reduce post-acidification of a fermented milk product comprising the deposited bacterium or a mutant thereof during storage after fermentation in comparison to a milk product comprising L. rhamnosus deposited as DSM 33515, wherein the reduction in post-acidification is determined after storing a product fermented with a starter culture and the L. rhamnosus strain over 21 days at 25° C.

In a related aspect the bacteria of the species L. rhamnosus deposited as DSM 34194 or mutants thereof exhibit the functional features described above, including:
(a) maintaining viability during storage at 25° C., wherein the test for maintaining viability comprises storing a fermented milk product comprising at least 1×10⁷ CFU/g of the bacterium before storage and wherein the fermented milk product comprises at least 1×10⁶ CFU/g of the bacterium after storage over 21 days at 25° C.;
(b) maintaining a higher total lactic acid cell count of a fermented milk product comprising the deposited bacterium or a mutant thereof during storage after fermentation in comparison to a milk product comprising L. rhamnosus deposited as DSM 33515, wherein the higher total lactic acid bacteria cell count is determined after storing a product fermented with a starter culture and the L. rhamnosus strain over 21 days at 25° C.; and/or
(c) reducing post-acidification of a fermented milk product comprising the deposited bacterium or a mutant thereof during storage after fermentation in comparison to a milk product comprising L. rhamnosus deposited as DSM 33515, wherein the reduction in post-acidification is determined after storing a product fermented with a starter culture and the L. rhamnosus strain over 21 days at 25° C.

In a further embodiment the present disclosure or application provides compositions comprising the bacterium of the species L. rhamnosus or mutant thereof as described above. The composition may contain other lactic acid bacteria organic and/or inorganic compounds. In one alternative, the composition comprises the bacterium and/or the mutant in a concentration of at least 10⁹ CFU/g, or in a concentration of at least 10¹⁰ CFU/g, or in a concentration of at least 10¹¹ CFU/g.

Respective compositions can have the form of a starter culture. As indicated above, starter cultures comprise lactic acid bacteria and are generally used to inoculate milk for producing fermented milk products. The starter culture can for example comprise bacteria of the genus Lactobacillus and/or Streptococcus, such as Lactobacillus delbrueckii subsp. bulgaricus and/or Streptococcus thermophilus.

The compositions of the present disclosure or application may further comprise cryoprotectants, lyoprotectants, antioxidants, nutrients fillers, flavorings, flavors and/or mixtures thereof. Respective compounds are often added to starter culture compositions for stabilizing the lactic acid bacteria during storage. The compositions the present disclosure or application may be maintained in frozen or freeze-dried state. Methods for generating frozen or freeze-dried compositions comprising viable lactic acid bacteria are well known in the art.

In particular, the use of protectants such as cryoprotectants and lyoprotectants for improving viability of lactic acid bacteria is well known to a person skilled in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorings.

In one aspect the present disclosure or application therefore provides compositions in the form of a solid frozen or freeze-dried starter culture comprising lactic acid bacteria in a concentration of at least 10⁹ colony forming units (CFU) per g of frozen material or in a concentration of at least 10¹⁰ CFU/g of frozen material or in a concentration of at least 10¹¹ CFU/g of frozen material.

In a further embodiment the present disclosure or application provides methods of producing fermented milk products comprising adding the bacterium of the species L. rhamnosus or the mutant thereof as described above or the composition as described above to a milk base and fermenting the milk base at a temperature between 22° C. and about 43° C. until a pH of 4.5 or less is reached or until a pH of 4.7 or less, such as 4.55 or less, is reached, or alternatively, until a total acidity (TA) of 70 or less is reached. Methods of fermenting milk products are well known in the art and a skilled person could implement respective methods using the bacteria and compositions of the present application.

In one preferred embodiment, the present disclosure or application provides a method of producing a fermented milk product, comprising adding the bacterium of the species L. rhamnosus or mutants thereof to a milk base comprising sugar such as sucrose, fructose or glucose, and fermenting the milk base at a temperature between 22° C. and about 43° C. until a pH of 4.5 or less is reached or until a pH of 4.7 or less, such as 4.55 or less, is reached.

The present disclosure or application also provides fermented milk products comprising the bacterium of the species L. rhamnosus or the mutant thereof as described above. The fermented milk product preferably contains the bacterium or the mutant thereof in a concentration of at least 10⁶ CFU/g. The fermented milk product can be obtainable by the method described above.

In a particularly preferred aspect, the fermented milk product does not exhibit a change in pH or a significant drop in cell count of the bacteria of the present disclosure or application during storage, even if stored at ambient temperatures. The fermented milk product can for example be characterized in that the product maintains a pH above 3.5 when stored for at least 21 days at 25° C. Alternatively or additionally, the fermented milk product can be characterized in that it maintains a viable lactic acid bacteria cell count of at least 10⁶ CFU/g when stored for at least 21 days at 25° C.

The present disclosure or application comprises any fermented milk product comprising the bacteria of the present disclosure or application. Preferably the fermented milk product is selected from yakult, cheese, yogurt, fruit yogurt, yogurt beverage, strained yogurt (Greek yogurt, Labneh, skyr), quark, fromage frais, sour cream, butter milk, white brined cheese, UF feta and cottage cheese, and/or cream cheese.

### Example 1—Use of DSM 34194 in Stirred Yogurt

In this example, fresh stirred yogurt containing high fructose corn syrup HFCS55 as added sugar was used to demonstrate the effect of DSM 34194. This type of recipe is widely used for affordable yogurt. In the reference sample, a common yogurt starter culture (Streptococcus thermophilus, Lactobacillus bulgaricus) was used as starter culture for yogurt fermentation. In the test sample, L. rhamnosus strain DSM 34194 was added to the yogurt starter culture to produce yogurt. Post-acidification and bacteria cell count were measured to evaluate the effect of DSM 34194.

### Samples:

Reference sample: YF-L907 (Streptococcus thermophilus, Lactobacillus bulgaricus), Chr Hansen A/S
Test sample: YF-L907+DSM 34194

The milk base was prepared according to Table 1 and pasteurized at 95° C. for 300 seconds.

TABLE 1
Milk base

| Ingredient | Supplier | Amount (w/w %) |
|---|---|---|
| Fresh milk | SanYuan (protein: 3.0%, fat: 3.4%) | 89% |
| Sucrose | TaiGu, refined sugar | 5% |
| HFCS55 | Cargill | 3% |
| Water | Nestle, purified water | 3% |

YF-L907 was inoculated at 100 u/T (units/ton) into milk base as reference sample and fermentation was carried out at 42° C. until the target pH 4.50 was reached. YF-L907 and DSM 34194 were inoculated at 100 u/T and 2.0E+6 CFU/g, respectively, into milk base as test sample, and fermented at 42° C. until the target pH 4.50 was reached and afterwards the curd was broken. The obtained yogurt was cooled to 25° C. and 2 bar pressure was applied to homogenize the texture. The yogurt was aseptically filled into individual cups and stored at 4-6° C. overnight.

To mimic breached cold chain storage, the matured yogurt was stored at 25° C. throughout the shelf life. The pH, total acidity (TA), and total lactic acid bacteria cell count were monitored every 7 days. Total lactic acid bacteria cell count was determined using Difco MRS agar and pour plate method with anaerobic incubation at 37° C. for 3 days. The L. rhamnosus cell count was determined using Difco MRS+vancomycin (50 mg/L) agar and pour plate method with anaerobic incubation at 37° C. for 3 days. MRS+vancomycin is a selective agar allowing growth of L. rhamnosus but not of Streptococcus thermophilus and Lactobacillus bulgaricus. The pH was measured at ambient temperature with Mettler Toledo PH meter (mode: SG2) and the pH meter was calibrated with two points (pH 4.01, pH 7.00) method before using. The total acidity (TA) was measured at ambient temperature according to method GB 5413.34 (National food safety standard Determination of acidity in milk and milk products, China).

### Results

TABLE 2
Fermentation result

| | Culture | Fermentation time | End pH | End TA |
|---|---|---|---|---|
| Reference | YF-L907 | 5 h 15 min | 4.49 | 67.76 |
| sample | | | | |
| Test sample | YF-L907 + | 5 h | 4.49 | 69.10 |
| | DSM 34194 | | | |

TABLE 3
pH at 25° C.

| | Culture | D + 0 | D + 1 | D + 7 | D + 14 | D + 21 |
|---|---|---|---|---|---|---|
| Reference | YF-L907 | 4.43 | 4.21 | 4.05 | 4.01 | 4.00 |
| sample | | | | | | |
| Test | YF-L907 + | 4.40 | 4.18 | 4.04 | 3.99 | 3.99 |
| sample | DSM 34194 | | | | | |

TABLE 4
Total acidity TA at 25° C.

| | Culture | D + 0 | D + 1 | D + 7 | D + 14 | D + 21 |
|---|---|---|---|---|---|---|
| Reference | YF-L907 | 67.76 | 80.04 | 92.82 | 92.54 | 93.56 |
| sample | | | | | | |
| Test | YF-L907 + | 69.10 | 82.10 | 95.26 | 94.60 | 95.46 |
| sample | DSM 34194 | | | | | |

TABLE 5
Cell count at 25° C., MRS agar

| | Culture | D + 0 | D + 7 | D + 14 | D + 21 |
|---|---|---|---|---|---|
| Reference | YF-L907 | 8.55E+8 | 8.80E+8 | 1.00E+6 | 0.00E+00 |
| sample | | | | | |
| Test | YF-L907 + | 1.01E+9 | 5.80E+8 | 6.00E+6 | 2.47E+6 |
| sample | DSM 34194 | | | | |

TABLE 6
Cell count at 25º C., MRS + vancomycin agar

| | Culture | D + 0 | D + 7 | D + 14 | D + 21 |
|---|---|---|---|---|---|
| Reference | YF-L907 | 0.00E+0 | 0.00E+0 | 0.00E+0 | 0.00E+0 |
| sample | | | | | |
| Test sample | YF-L907 + | 3.10E+6 | 5.20E+6 | 4.60E+6 | 2.51E+6 |
| | DSM 34194 | | | | |

At 25° C., both reference sample and test sample showed pH decreasing and TA increasing. The test sample maintained similar values as the reference sample for both pH and TA, showing that DSM 34194 generated little acidification. Therefore, the test sample showed similar post acidification as the reference sample.

At 25° C., total lactic acid bacteria cell count in the reference sample was ≤1.0E+6 CFU/g after 14 days and drastically decreased on day 21. The cell count enumerated was 0.00E+0 CFU/g on day 21 (single step dilution was used in enumeration; actual value may be slightly higher than 0.00E+0 CFU/g but nevertheless much lower than 1.0E+6 CFU/g). This meant that at challenged storage condition, the yogurt starter culture Streptococcus thermophilus and Lactobacillus bulgaricus decline rapidly. However, in the test sample, the total lactic acid bacteria cell count stayed above 1.0E+6 CFU/g for 21 days, fully meeting the requirements of Chinese national standard. The cell count in reference sample and test sample was checked with MRS+vancomycin agar. In the reference sample, no bacteria were found, which means all Streptococcus thermophilus and Lactobacillus bulgaricus were inhibited by vancomycin; in the test sample, the cell count was above 1.0E+6 CFU/g for 21 days and the cell count value close to the value in the reference sample.

This demonstrates that DSM 34194 survives well at ambient temperature and contributes to total lactic acid bacteria cell count with little or no impact on post-acidification. In addition, DSM 34194 can be advantageously used in fermented milk products containing syrup recipe.

### Example 2

### Use of DSM 34194 in White Yakult

In this example, white yakult style product was used to demonstrate the effect of DSM 34194. White yakult is a two-step process beverage where the first step is to produce fermented milk base and the second step is to dilute the fermented milk base with pasteurized syrup. As reference sample, normal yogurt culture (Streptococcus thermophilus, Lactobacillus delbrueckii subsp. bulgaricus) was used as starter culture for fermentation. For the test sample, the L. rhamnosus strain DSM 34194 was added to the normal yogurt culture. Post-acidification and bacteria cell count was measured to evaluate the effect of DSM 34194.

### Samples:

Reference sample: Premium1.0, Chr. Hansen A/S
Test sample: Premium1.0+DSM 34194

The milk base was prepared according to Table 7 and pasteurized at 95° C. for 300 seconds.

TABLE 7
Milk base

| Ingredients | Supplier | Amount (w/w %) |
|---|---|---|
| Skimmed Milk Powder | NZMP | 11.2% |
| Sucrose | Taigu | 5.0% |
| Water | Nestle | 83.8% |

Premium1.0 was inoculated at 100 u/T (units/ton) into milk base as a reference sample and fermentation was carried out at 42° C. until the target pH 4.30 was reached. Premium1.0 and DSM 34194 were inoculated at 100 u/T and 5.0E+6 CFU/g, respectively, into milk base as test sample, and fermented at 42° C. until the target pH 4.30 was reached and afterwards the curd was broken. The obtained yogurt base was cooled to below 15° C. Syrup water was prepared according to Table 8 recipe and pasteurization at 95° C. for 300 s was performed. Pasteurized syrup water was cooled to below 15° C. Fermented milk base was mixed with the syrup water according to Table 9 recipe and afterwards homogenized at 50 bar and then 150 bar at below 15° C. The finished beverage was then filled into individual bottles and stored at 4-6° C. overnight.

TABLE 8
Syrup water

| Ingredients | Supplier | Amount (w/w %) |
|---|---|---|
| Fructose | Cargill | 6.17% |
| Sucrose | Taigu | 1.85% |
| Pectin 150H | CP Kelco | 0.37% |
| Citric acid | | 0.25% |
| Sodium citric acid | | 0.05% |
| Water | | 91.31% |

TABLE 9
White yakult style beverage recipe

| Ingredients | Amount (w/w %) |
|---|---|
| Fermented base | 19% |
| Syrup water | 81% |

To mimic broken cold chain storage, the white yakult style beverage was stored at 25° C. The pH, total acidity (TA) and total lactic acid bacteria cell count was monitored every 7 days.

Total lactic acid bacteria cell count was determined by using Difco MRS agar and pour plate method with anaerobic incubation at 37° C. for 3 days. The L. rhamnosus cell count was determined using Difco MRS+vancomycin (50 mg/L) agar and pour plate method with anaerobic incubation at 37° C. for 3 days. The pH was measured at ambient temperature with Mettler Toledo pH meter (mode: SG2). The PH meter was calibrated with two points (pH 4.01, pH 7.00) method before using. The TA (total acidity) was measured at ambient temperature according to method GB 5413.34 (National food safety standard Determination of acidity in milk and milk products, China).

### Results

TABLE 10
Fermentation result

| | | Fermentation | | |
|---|---|---|---|---|
| | Culture | time | End pH | End TA |
| Reference | Premium1.0 | 8 h | 4.35 | 86.78 |
| sample | | | | |
| Test sample | Premium + | 8 h | 4.36 | 87.12 |
| | DSM 34194 | | | |

TABLE 11
Cell count in fermented milk base

| | | | MRS + |
|---|---|---|---|
| | Culture | MRS agar | Vancomycin agar |
| Reference | Premium1.0 | 7.45E+8 | 0.00E+0 |
| sample | | | |
| Test sample | Premium1.0 + | 1.14E+9 | 1.46E+7 |
| | DSM 34194 | | |

TABLE 12
pH at 25° C.

| | Culture | D + 0 | D + 7 | D + 14 | D + 21 |
|---|---|---|---|---|---|
| Reference | Premium1.0 | 3.70 | 3.62 | 3.55 | 3.52 |
| sample | | | | | |
| Test sample | Premium1.0 + | 3.69 | 3.60 | 3.54 | 3.52 |
| | DSM 34194 | | | | |

TABLE 13
Total acidity (TA) at 25° C.

| | Culture | D + 0 | D + 7 | D + 14 | D + 21 |
|---|---|---|---|---|---|
| Reference | Premium1.0 | 44.38 | 48.24 | 49.52 | 50.46 |
| sample | | | | | |
| Test sample | Premium1.0 + | 45.50 | 49.64 | 50.78 | 52.74 |
| | DSM 34194 | | | | |

TABLE 14
Cell count at 25º C., MRS agar

| | Culture | D + 0 | D + 7 | D + 14 | D + 21 |
|---|---|---|---|---|---|
| Reference | Premium1.0 | 1.11E+8 | 2.08E+6 | 0.00E+0 | 0.00E+0 |
| sample | | | | | |
| Test sample | Premium1.0 + | 1.40E+8 | 7.10E+6 | 1.12E+7 | 4.10E+7 |
| | DSM 34194 | | | | |

TABLE 15
Cell count at 25º C., MRS + vancomycin agar

| | Culture | D + 0 | D + 7 | D + 14 | D + 21 |
|---|---|---|---|---|---|
| Reference | Premium1.0 | 0.00E+0 | 0.00E+0 | 0.00E+0 | 0.00E+0 |
| sample | | | | | |
| Test sample | Premium1.0 + | 1.95E+6 | 4.60E+6 | 1.10E+7 | 4.50E+7 |
| | DSM 34194 | | | | |

At 25° C., both reference sample and test sample showed pH decreasing and TA increasing. The test sample maintained similar values as the reference sample for both pH and TA, showing that DSM 34194 generated little acidification. Therefore, the test sample showed the similar post-acidification as the reference sample.

At 25° C., total lactic acid bacteria cell count in the reference sample decreased drastically after 14 days, unable to meet 1.0E+6 CFU/g required by national standard, for example in China. The cell count enumerated was 0.00E+0 CFU/g on day 14 and day 21 (single step dilution was used in enumeration; actual value may be slightly higher than 0.00E+0 CFU/g but nevertheless much lower than 1.0E+6 CFU/g). However, in the test sample where DSM 34194 was added, the total lactic acid bacteria cell count stayed above 1.0E+6 CFU/g for 21 days, fully meeting the requirements of Chinese national standard. The cell count in the reference sample was checked with MRS+vancomycin agar. In the reference sample, no bacteria were found, which means all Streptococcus thermophilus and Lactobacillus delbrueckii subsp. bulgaricus were inhibited by vancomycin. In the test sample, the cell count was above 1.0E+6 CFU/g on both MRS agar and MRS+vancomycin agar for 21 days, meaning that after 14 days storage, it was DSM 34194 which was still alive in beverage.

This demonstrates that DSM 34194 survives well at ambient temperature and contributes to total lactic acid bacteria cell count with little impact on post-acidification.

### Example 3

Comparison of DSM 34194 and Lactobacillus rhamnosus Strain LGG® Deposited as ATCC53103

In this example, a comparison was carried out between different strains of Lactobacillus rhamnosus, in particular Lactobacillus rhamnosus strain LGG® deposited as ATCC53103 (LGG®, reference U.S. Pat. No. 10,072,310) or Lactobacillus rhamnosus DSM 34194 with respect to post-acidification and cell count stability during long-term storage at 25° C. for a lactose-deficient culture (such as YoFlex Acidifix 1.0) and a lactose-positive culture (such as YoFlex Premium 1.0), in yogurt prepared with 8% sucrose and stored at 25° C.

### Samples:

Reference sample: YoFlex Acidifix 1.0 (lactose-deficient culture containing lactose-deficient Streptococcus thermophilus strains and lactose-deficient Lactobacillus delbrueckii subsp. bulgaricus strains), Chr. Hansen A/S
Test sample: YoFlex Acidifix 1.0+LGG® (Lactobacillus rhamnosus strain LGG®, deposited as ATCC53103), both commercialized by Chr. Hansen A/S
Test sample: YoFlex Acidifix 1.0+DSM 34194
Reference sample: YoFlex Premium 1.0 (lactose-positive culture containing lactose-deficient Streptococcus thermophilus strains and lactose-positive Lactobacillus delbrueckii subsp. bulgaricus strains), Chr. Hansen A/S
Test sample: YoFlex Premium 1.0+LGGR (Lactobacillus rhamnosus strain LGG®, deposited as ATCC53103), both Chr. Hansen A/S Test sample: YoFlex Premium 1.0+DSM 34194

Milk base was prepared according to Table 16 and pasteurized at 95° C. for 300 seconds.

TABLE 16
Milk base

| | | Composition of milk base | | |
|---|---|---|---|---|
| Ingredient | Amount | Protein | Carbohydrate | Fat |
| 3.5% fat | 14000 g | 4.0% | 4.8% | 3.5% |
| 38% fat | 391 g | 2.4% | 2.8% | 38.5% |
| Water | 5079 g | 0.0% | 0.0% | 0.0% |
| Sucrose | 1693 g | 0.0% | 100% | 0.0% |
| Milk base with 8% sucrose | 21163 g | 2.67% | 11.25% | 3.00% |

The milk base was inoculated with a reference sample (500 U/2500 L of YoFlex Acidifix 1.0 or 250 U/2500 L of YoFlex Premium 1.0) with or without addition of 4E+6 CFU/g of LGG® or DSM 34194. Milk fermentation was carried out in 200 ml bottles at 43° C. until end pH of 4.55 was reached to produce yogurt. After fermentation the yogurts were stored at 25° C. pH was measured after 1, 14 and 28 days.

Cell count evaluation was made after 1, 14 and 28 days by spreading yogurts on MRS+vancomycin and MRS without vancomycin and incubating plates anaerobically at 37° C.

### Results

TABLE 17
pH at 25° C.

| | Culture | Day 1 | Day 14 | Day 28 |
|---|---|---|---|---|
| Reference sample | Acidifix | 4.28 | 3.83 | 3.70 |
| Test sample | Acidifix + LGG ® | 4.27 | 3.85 | 3.75 |
| Test sample | Acidifix + DSM | 4.23 | 4.09 | 3.98 |
| | 34194 | | | |
| Reference sample | Premium | 4.20 | 3.95 | 3.77 |
| Test sample | Premium + LGG ® | 4.19 | 3.70 | 3.62 |
| Test sample | Premium + DSM | 4.23 | 4.02 | 4.02 |
| | 34194 | | | |

As seen in Table 17, DSM 34194 gives significantly higher pH and simultaneously a more stable pH when yogurts stored at 25° C. than yogurts made with LGG® or without a cell count stable culture (reference samples), both in yogurts made with starter culture Acidifix and Premium1.0. Therefore, DSM 34194 has significantly less impact in post-acidification than LGG®, preferably after day 1, such as between days 2 and 28 or between days 14 and 28.

In addition, cell counts were also determined.

TABLE 18
Cell count at 25º C., MRS agar

| | | Day 1 | Day 14 | Day 28 |
|---|---|---|---|---|
| Reference sample | Acidifix | 3.7E+08 | 3.4E+08 | 3.4E+08 |
| Test sample | Acidifix + LGG ® | 3.9E+08 | 3.8E+08 | 3.4E+08 |
| Test sample | Acidifix + DSM 34194 | 5.1E+08 | 5.3E+07 | 5.1E+07 |
| Reference sample | Premium | 5.8E+08 | 2.7E+07 | 2.7E+05 |
| Test sample | Premium + LGG ® | 5.0E+08 | 3.8E+08 | 2.7E+08 |
| Test sample | Premium + DSM 34194 | 4.8E+08 | 1.6E+07 | 4.4E+06 |

Cells counts decreased to below 1E+6 CFU/g in yogurt made with Premium without a cell count stable culture after 28 days. In samples with LGG® or DSM 34194 the cell counts were above 1E+6 CFU/g after 28 days.

TABLE 19
Cell count at 25° C., MRS + vancomycin agar

| | | Day 1 | Day 14 | Day 28 |
|---|---|---|---|---|
| Reference sample | Acidifix | <50 | <50 | <50 |
| Test sample | Acidifix + LGG ® | 6.9E+07 | 4.1E+07 | 2.0E+07 |
| Test sample | Acidifix + DSM 34194 | 1.4E+07 | 4.7E+06 | 2.9E+06 |
| Reference sample | Premium | <50 | <50 | <50 |
| Test sample | Premium + LGG | 6.6E+07 | 3.6E+07 | 1.9E+07 |
| Test sample | Premium + DSM 34194 | 1.7E+07 | 1.3E+07 | 5.0E+06 |

In reference samples spread on MRS+vancomycin no growth was detected, confirming that all Streptococcus thermophilus and Lactobacillus delbrueckii subsp. bulgaricus were inhibited by vancomycin. In test samples with DSM 34194 or LGG® added, the cell count was above 1.0E+6 CFU/g on both MRS agar and MRS+vancomycin agar for up to 28 days.

In conclusion, examples 1 and 2 show that regardless of the starter cultures used (YF-L907 or Premium 1.0, both from Chr. Hansen A/S) or the fermented milk product made (stirred yogurt or white yakult) DSM 34194 survives well at ambient temperature and contributes to total lactic acid bacteria cell count with little or no impact on post-acidification. Example 3 shows an unexpected technical effect of DSM 34194 over LGG®. Even though both DSM 34194 and LGG® are L. rhamnosus strains, example 3 clearly shows that DSM 34194 has a different and desirable post-acidification profile versus LGG® and is, therefore, preferred for the purposes of the present disclosure or invention, which is to have high cell count (≥1E+6 CFU/g) in fermented milk products during storage even at ambient temperature while simultaneously having a low post-acidification activity.

### Deposit and Expert Solution

The applicant has made the following deposits at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures Inhoffenstr. 7B, 38124 Braunschweig, Germany.

| Strain | Accession No. | Deposit date |
|---|---|---|
| L. rhamnosus | DSM 34194 | 2022 Mar. 1 |

The applicant requests that a sample of the deposited microorganisms stated below may only be made available to an expert, subject to available provisions governed by Industrial Property Offices of States Party to the Budapest Treaty, until the date on which the patent is granted.

### LIST OF EMBODIMENTS IN CLAIM FORMAT

1. A bacterium of the species L. rhamnosus deposited as DSM 34194 or a mutant thereof.

2. The bacterium of the species L. rhamnosus of embodiment 1, wherein the bacterium is a mutant of the bacterium of the species L. rhamnosus deposited as DSM 34194, wherein the mutant maintains viability during storage at 25° C., and wherein the test for maintaining viability comprises storing a fermented milk product comprising at least 1×10⁷ CFU/g of the mutant before storage and wherein the fermented milk product comprises at least 1×10⁶ CFU/g of the mutant after storage over 21 days at 25° C.

3. A composition comprising the bacterium of the species L. rhamnosus according to embodiment 1 or 2 or mutant thereof.

4. The composition of embodiment 3, wherein the bacterium or mutant is in a concentration of at least 10⁹ CFU/g, or in a concentration of at least 10¹⁰ CFU/g, or in a concentration of at least 10¹¹ CFU/g.

5. The composition of embodiment 3 or 4, wherein the composition further comprises a starter culture.

6. The composition of any of embodiments 3 to 5, wherein the starter culture comprises bacteria of the species Lactobacillus delbrueckii subsp. bulgaricus and/or Streptococcus thermophilus.

7. The composition of any of embodiments 3 to 6, wherein the composition further comprises cryoprotectants, lyoprotectants, antioxidants and/or nutrients.

8. The composition of any of embodiments 3 to 7, wherein the composition is frozen or freeze-dried.

9. A method of producing a fermented milk product comprising adding the bacterium of the species L. rhamnosus or mutant thereof of any of embodiments 1 to 4 or the composition of any of embodiments 5 to 8 to a milk base and fermenting the milk base at a temperature between 22° C. and about 43° C. until a pH of 4.5 or less or a total acidity (TA) of 70 or less is reached or until a pH of 4.7 or less, such as 4.55 or less, is reached and thereby obtaining the fermented milk product.

10. The method of embodiment 9, wherein the milk base has 4-12% (w/w) of sucrose, preferably 6-10% (w/w) or 8-9% (w/w) of sucrose.

11. The method of any of embodiments 9 or 10, wherein the bacterium of the species L. rhamnosus or mutant thereof of any of embodiments 1 to 4 or the composition of any of embodiments 5 to 8 is added in a concentration of at least 1×10⁶ CFU/g, preferably 1×10⁶ CFU/g to 7×10⁶ CFU/g, more preferably 2×10⁶ CFU/g to 6×10⁶ CFU/g or 4×10⁶ CFU/g to 5×10⁶ CFU/g.

12. A fermented milk product comprising the bacterium of the species L. rhamnosus according to embodiment 1 or 2 or the mutant thereof.

13. The fermented milk product of embodiment 12, wherein the bacterium or the mutant thereof is present in a concentration of at least 10⁶ CFU/g, preferably in a concentration of 10⁶ CFU/g to 10⁹ CFU/g or 10⁶ CFU/g to 10⁸ CFU/g or 10⁶ CFU/g to 10⁷ CFU/g.

14. The fermented milk product of embodiment 12 or 13, wherein the fermented milk product is obtainable by the method of any of the embodiments 9 to 11.

15. The fermented milk product of any of embodiments 12 to 14, wherein the fermented milk product maintains a pH above 3.5, preferably above 3.8, when stored for at least 21 days at 25° C.

16. The fermented milk product of any of embodiments 12 to 15, wherein the fermented milk product maintains a viable lactic acid bacteria cell count of at least 10⁶ CFU/g when stored for at least 21 days at 25° C.

17. The fermented milk product of any of embodiments 12 to 16, wherein the fermented milk product is yakult, cheese, yogurt, fruit yogurt, yogurt beverage, strained yogurt, such as Greek yogurt or Labneh, quark, fromage frais or cream cheese.

1. A bacterium of the species Lacticaseibacillus (L.) rhamnosus deposited as DSM 34194.

2. A composition comprising the bacterium of the species L. rhamnosus according to claim 1.

3. The composition of claim 2, wherein the bacterium is in a concentration of at least 10⁹ CFU/g, or in a concentration of at least 10¹⁰ CFU/g, or in a concentration of at least 10¹¹ CFU/g.

4. The composition of claim 2, wherein the composition further comprises a starter culture, wherein the starter culture comprises bacteria of the species Lactobacillus delbrueckii subsp. bulgaricus and/or Streptococcus thermophilus.

5. The composition of claim 2, wherein the composition further comprises cryoprotectants, lyoprotectants, antioxidants and/or nutrients.

6. The composition of claim 2, wherein the composition is frozen or freeze-dried.

7. A method of producing a fermented milk product comprising adding the bacterium of the species L. rhamnosus of claim 1 to a milk base and fermenting the milk base at a temperature between 22° C. and about 43° C. until a pH of 4.5 or less or a total acidity (TA) of 70 or less is reached or until a pH of 4.7 or less, such as 4.55 or less, is reached.

8. The method of claim 7, wherein the milk base has 4-12% (w/w) of sucrose, 6-10% (w/w) of sucrose, or 8-9% (w/w) of sucrose.

9. The method of claim 7, wherein the bacterium of the species L. rhamnosus deposited as DSM 34197 is added in a concentration of at least 1×10⁶ CFU/g, 1×10⁶ CFU/g to 7×10⁶ CFU/g, 2×10⁶ CFU/g to 6×10⁶ CFU/g, or 4×10⁶ CFU/g to 5×10⁶ CFU/g.

10. A fermented milk product comprising the bacterium of the species L. rhamnosus according to claim 1.

11. The fermented milk product of claim 10, wherein the bacterium is present in a concentration of at least 10⁶ CFU/g, 10⁶ CFU/g to 10⁹ CFU/g, 10⁶ CFU/g to 10⁸ CFU/g, or 10⁶ CFU/g to 10⁷ CFU/g.

12. A fermented milk product produced, by the method of claim 7.

13. The fermented milk product of claim 10, wherein the fermented milk product maintains a pH above 3.5 or preferably above 3.8 when stored for at least 21 days at 25° C.

14. The fermented milk product of claim 10, wherein the fermented milk product maintains a viable lactic acid bacteria cell count of at least 10⁶ CFU/g when stored for at least 21 days at 25° C.

15. The fermented milk product of claim 10, wherein the fermented milk product is yakult, cheese, yogurt, fruit yogurt, yogurt beverage, strained yogurt such as Greek yogurt or Labneh, quark, fromage frais, or cream cheese.
